(19) United States
(12) Patent Application Publication
(10) Pub. No.: US 2013/0225517 A1
(43) Pub. Date: Aug. 29, 2013

(54) Therapeutic Compounds

(71) Applicant: University of Iowa Research Foundation, (US)

(72) Inventors: Charles M. Brenner, Iowa City, IA (US); Rebecca L. Fagan, Iowa City, IA (US)

(73) Assignee: University of Iowa Research Foundation, Iowa City, IA (US)

(21) Appl. No.: 13/774,594

(22) Filed: Feb. 22, 2013

Related U.S. Application Data
(60) Provisional application No. 61/603,121, filed on Feb. 24, 2012.

(51) Int. Cl. C07C 233/51; A61K 31/165; A61K 31/085; A61K 31/4184; A61K 31/122; A61K 31/352; A61K 31/194; A61K 31/7048; A61K 45/06; A61K 31/704

(52) U.S. Cl. CPC C07C 233/51; A61K 45/06; A61K 31/165; A61K 31/085; A61K 31/704; A61K 31/122; A61K 31/352; A61K 31/194; A61K 31/7048; A61K 31/4184
USPC 514/31; 546/102; 546/103; 546/105; 552/208; 552/236; 552/237; 552/238; 552/240; 552/241; 552/244; 552/265; 514/297; 514/680; 514/656; 514/569; 514/567; 514/566; 514/564; 514/33; 514/721; 514/456; 514/681; 514/574; 514/407; 514/563; 435/375

(57) ABSTRACT

The invention provides compounds of formula I:

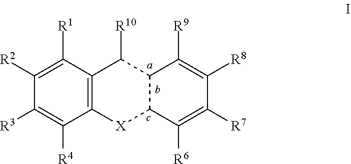

or salts thereof as described herein. The invention also provides pharmaceutical compositions comprising a compound of formula I and therapeutic methods for treating cancer.

### PRIORITY OF INVENTION

This application claims priority to U.S. Provisional Application No. 61/603,121 that was filed on Feb. 24, 2012. The entire content of this provisional application is hereby incorporated herein by reference.

### GOVERNMENT FUNDING

The invention described herein was made with government support under Contract Number HHSN261200433000C (Subcontract to University of Toledo, Work Assignment 14). The United States Government has certain rights in the invention.

### BACKGROUND OF THE INVENTION

There are several isoforms in the DNA methyltransferase family across species, but they all catalyze the transfer of a methyl group (from donor S-adenosyl methionine aka AdoMet) to DNA. The methyl group from AdoMet is typically transferred to the fifth carbon position of cytosine (cytosine-5 or C5), mostly within CpG dinucleotides. This methylation, together with histone modifications, plays an important role in modulating chromatin structure, thus controlling gene expression and many other chromatin-dependent processes. The various isoforms are stimulated to perform this function by a variety of signaling pathways to produce a distinct set of resulting methylations.

There are four known DNA methyltransferases that play a role in human somatic cell biology, DNA methyltransferase 1 and splice variant DNMT 1b (DNMT 1 & DNMT 1b) and DNA methyltransferase 3 a & b (DNMT 3a & DNMT 3b). For the purpose of categorization, the DNMTs can be grouped according to de novo vs. maintenance activity. De novo methyltransferases are enzymes that are able to methylate previously unmethylated CpG sequences, and maintenance methyltransferases copy pre-existing methylation marks onto new DNA strands during replication. DNMT1 is the most abundant DNA methyltransferase in mammalian cells and predominatly methylates hemimethylated CpG dinucleotides as a maintenance methyltransferase, but DNMT1 has also been shown to function as a de novo DNA methyltransferase.

The result of DNA methylation, like that of histone methylation, is to make that portion of the genomic DNA inaccessible to transcription factors and prevent expression of the gene. This is a normal part of DNA maintenance, preventing expression of unnecessary or unwanted genes during most of the life of the cell. This activity has been shown to be key in inheritance and DNA regions methylated in the mother are quickly mirrored in progeny. This powerful tool for totally blocking the expression of a gene has been shown to also play an important role in cancer development and progression. DNA housekeeping genes and tumor suppressor genes that haven't been mutated in earlier stages of cancer progression are often methylated (unlike their wt tissue counterparts) to prevent their expression and potential role in rescuing the cell from its progression into dysplasia and neoplasia. The importance that DNMTs play in cancer progression has made them a recent target for anti-cancer drug development.

Although advances in cancer treatment have occurred over the past 20 years, there remains a need for agents or methods that are useful for treating or preventing cancer.

### SUMMARY OF THE INVENTION

Applicant has discovered that certain anthracene-related compounds are inhibitors of DNMT1 activity and may thus be useful as anticancer therapeutics.

In one embodiment the invention provides a compound of formula I:

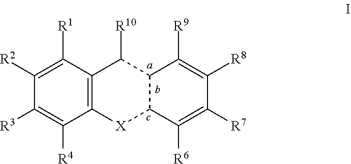

wherein:

X is N or CR⁵, the dashed bonds labeled a and c are double bonds and the dashed bond labeled b is a single bond; or X is CR⁵, the dashed bonds labeled a and c are single bonds, the dashed bond labeled b is a double bond and the substituents R⁵ and R¹⁰ are oxo (═O) groups;

R¹ is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more (e.g. 1, 2, 3, 4 or 5) Z¹ groups;

R² is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more (e.g. 1, 2, 3, 4 or 5) Z¹ groups;

R³ is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more (e.g. 1, 2, 3, 4 or 5) Z¹ groups;

R⁴ is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more (e.g. 1, 2, 3, 4 or 5) Z¹ groups;

R⁵ is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more (e.g. 1, 2, 3, 4 or 5) Z¹ groups;

R⁶ is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more (e.g. 1, 2, 3, 4 or 5) Z¹ groups;

R⁷ is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more (e.g. 1, 2, 3, 4 or 5) Z¹ groups;

R⁸ is H, halo, (Cⱼ—C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more (e.g. 1, 2, 3, 4 or 5) Z¹ groups;

R⁹ is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more (e.g. 1, 2, 3, 4 or 5) Z¹ groups;

R¹⁰ is H, H halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more Z¹ groups;

each Z¹ is independently selected from halo, (C₁-C₆)alkyl, —O(C₁-C₆)alkyl, NRₐR_{b}, OH and CO₂H, wherein (C₁-C₆)alkyl is optional substituted with one or more (e.g. 1, 2, 3, 4 or 5) groups selected from halo, —O(C₁-C₆)alkyl, NR_{c}R_{d}, NRₑC(═O)R_{f}, OH and CO₂H;

Rₐ and R_{b} are each independently H or (C₁-C₆)alkyl, or Rₐ and R_{b} together with the nitrogen to which they are attached form a heterocycle;

R_{c} and R_{d} are each independently H or (C₁-C₆)alkyl, or R_{c} and R_{d} together with the nitrogen to which they are attached form a heterocycle;

each Rₑ is independently H or (C₁-C₆)alkyl; and

each R_{f} is independently H or (C₁-C₆)alkyl; or a salt thereof.

The invention also provides a pharmaceutical composition comprising a compound of formula I or a pharmaceutically acceptable salt thereof and a pharmaceutically acceptable carrier.

The invention also provides a pharmaceutical composition comprising a compound of formula II-XI:

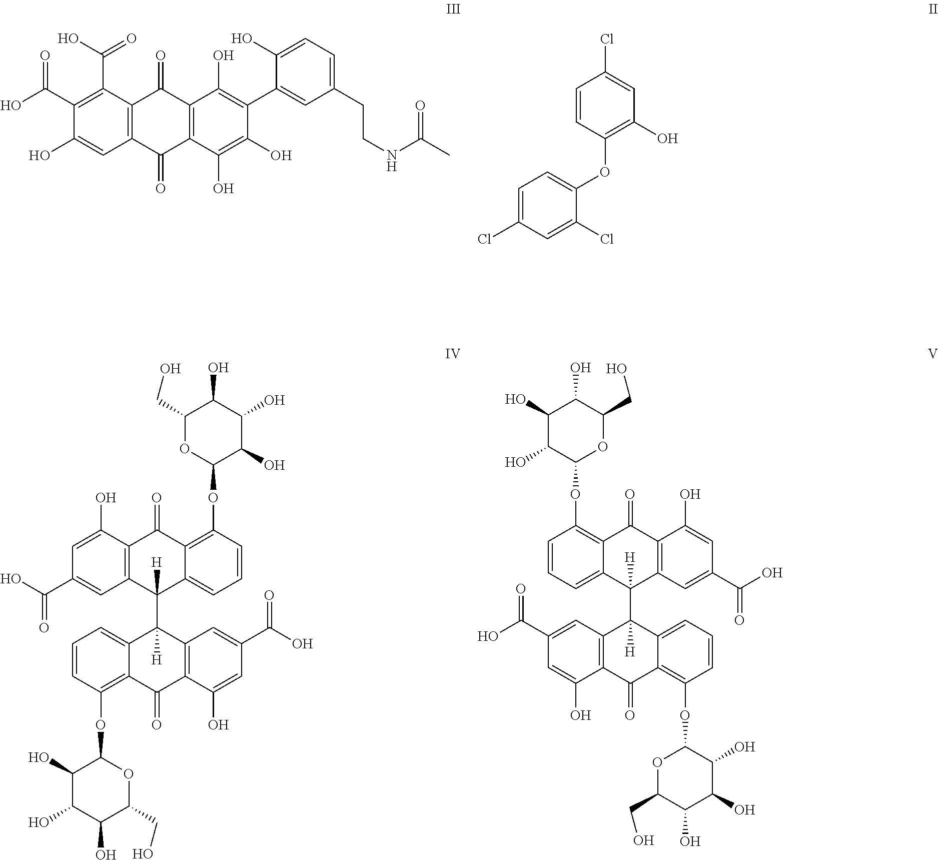

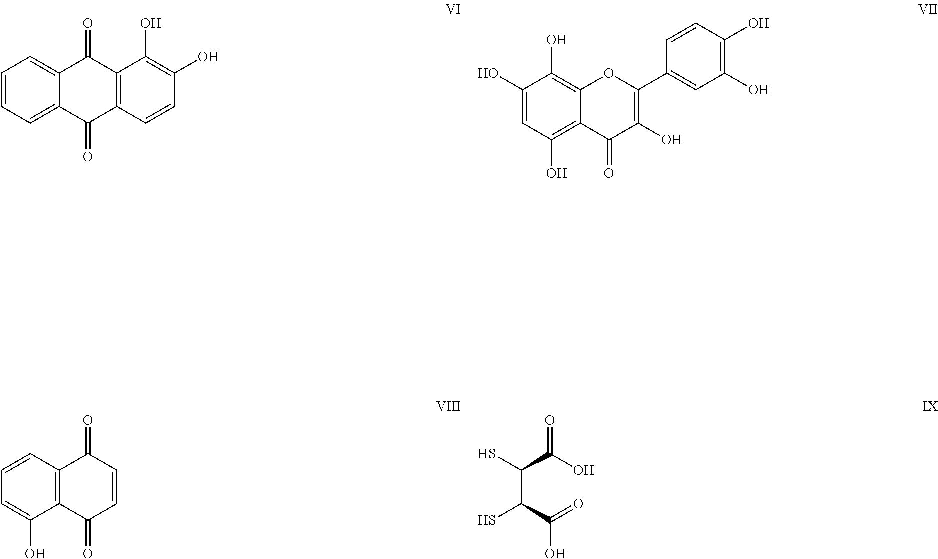 [SMILES: O=C1c2ccccc2C(=O)c2c1ccc(O)c2O ; O=c1c(O)c(-c2ccc(O)c(O)c2)oc2c(O)c(O)cc(O)c12 ; O=C1C=CC(=O)c2c(O)cccc21 ; O=C(O)[C@@H](S)[C@@H](S)C(=O)O]

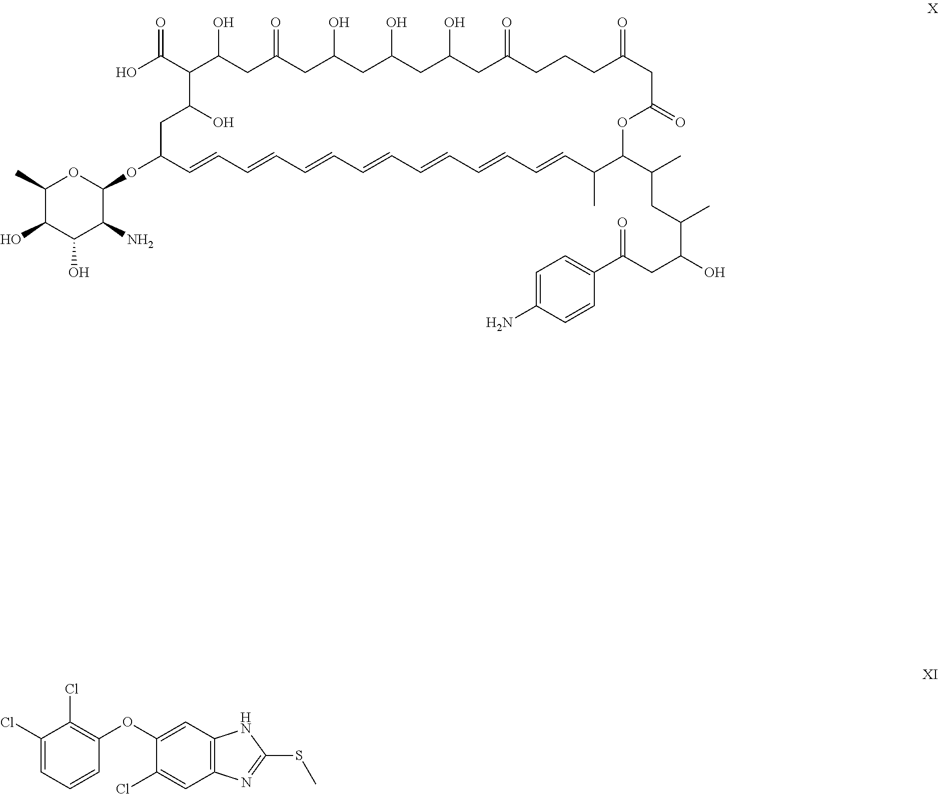 [SMILES: CC1/C=C/C=C/C=C/C=C/C=C/C=C/C=C/C(O[C@@H]2O[C@H](C)[C@H](O)[C@@H](O)[C@@H]2N)CC(O)C(C(=O)O)C(O)CC(=O)CC(O)CC(O)CC(O)CC(=O)CCCC(=O)CC(=O)OC1C(C)CC(C)C(O)CC(=O)c1ccc(N)cc1 ; CSc1nc2cc(Cl)c(Oc3cccc(Cl)c3Cl)cc2[nH]1]

or a pharmaceutically acceptable salt thereof, and a pharmaceutically acceptable carrier.

The invention also provides a method for suppressing cancer cell (e.g. blood cancer cell or solid tumor cancer cell) growth comprising contacting (in vitro or in vivo) the cancer cell with a compound of formula I-XI or a salt thereof.

The invention also provides a method for inhibiting DNMT in a cell (e.g. a blood cancer cell or a solid tumor cancer cell), comprising contacting the cell in vitro or in vivo with an effective amount of a compound of formula I-XI or a salt thereof.

The invention also provides a method for treating cancer (e.g. malignancies of the blood system such as myelodysplatic syndromes or solid tumors) in a mammal (e.g. a human) comprising administering the mammal an effective amount of a compound of formula I-XI or a pharmaceutically acceptable salt thereof.

The invention also provides a compound of formula I-XI or a pharmaceutically acceptable salt thereof for use in medical therapy.

The invention also provides the use of a compound of formula I-XI or a pharmaceutically acceptable salt thereof for the manufacture of a medicament useful for the treatment of or prevention of cancer (e.g. malignancies of the blood system such as myelodysplatic syndromes or solid tumors) in a mammal (e.g. a human).

The invention also provides a compound of formula I-XI or a pharmaceutically acceptable salt thereof for use in the prophylactic or therapeutic treatment of cancer (e.g. malignancies of the blood system such as myelodysplatic syndromes or solid tumors).

The invention also provides processes and intermediates disclosed herein that are useful for preparing compounds of formula I or salts thereof.

### DETAILED DESCRIPTION

The following definitions are used, unless otherwise described: halo or halogen is fluoro, chloro, bromo, or iodo. Alkyl, alkoxy, alkenyl, alkynyl, etc. denote both straight and branched groups; but reference to an individual radical such as propyl embraces only the straight chain radical, a branched chain isomer such as isopropyl being specifically referred to.

The term “aryl” as used herein refers to a single aromatic ring or a multiple condensed ring system. For example, an aryl group can have 6 to 20 carbon atoms, 6 to 14 carbon atoms, or 6 to 12 carbon atoms. Aryl includes a phenyl radical. Aryl also includes multiple condensed ring systems (e.g. ring systems comprising 2, 3 or 4 rings) having about 9 to 20 carbon atoms in which at least one ring is aromatic. Such multiple condensed ring systems may be optionally substituted with one or more (e.g. 1, 2 or 3) oxo groups on any carbocycle portion of the multiple condensed ring system. It is to be understood that the point of attachment of a multiple condensed ring system, as defined above, can be at any position of the ring system including an aryl or a carbocycle portion of the ring. Typical aryl groups include, but are not limited to, phenyl, indenyl, naphthyl, 1, 2, 3, 4-tetrahydronaphthyl, anthracenyl, and the like.

The term “heteroaryl” as used herein refers to a single aromatic ring or a multiple condensed ring system. The term includes single aromatic rings of from about 1 to 6 carbon atoms and about 1-4 heteroatoms selected from the group consisting of oxygen, nitrogen and sulfur in the rings. The sulfur and nitrogen atoms may also be present in an oxidized form provided the ring is aromatic. Such rings include but are not limited to pyridyl, pyrimidinyl, oxazolyl or furyl. The term also includes multiple condensed ring systems (e.g. ring systems comprising 2, 3 or 4 rings) wherein a heteroaryl group, as defined above, can be condensed with one or more heteroaryls (e.g. naphthyridinyl), heterocycles, (e.g. 1, 2, 3, 4-tetrahydronaphthyridinyl), carbocycles (e.g. 5,6,7,8-tetrahydroquinolyl) or aryls (e.g. indazolyl) to form a multiple condensed ring system. Such multiple condensed ring systems may be optionally substituted with one or more (e.g. 1, 2, 3 or 4) oxo groups on the carbocycle or heterocycle portions of the condensed ring. It is to be understood that the point of attachment of a multiple condensed ring system (as defined above for a heteroaryl) can be at any position of the multiple condensed ring system including a heteroaryl, heterocycle, aryl or carbocycle portion of the multiple condensed ring system and at any suitable atom of the multiple condensed ring system including a carbon atom and heteroatom (e.g. a nitrogen). Exemplary heteroaryls include but are not limited to pyridyl, pyrrolyl, pyrazinyl, pyrimidinyl, pyridazinyl, pyrazolyl, thienyl, indolyl, imidazolyl, oxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl and thianaphthenyl.

The term “heterocyclyl” or “heterocycle” as used herein refers to a single saturated or partially unsaturated ring or a multiple condensed ring system. The term includes single saturated or partially unsaturated rings (e.g. 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g. 1, 2 or 3) oxo groups and the sulfur and nitrogen atoms may also be present in their oxidized forms. Such rings include but are not limited to azetidinyl, tetrahydrofuranyl or piperidinyl. The term “heterocycle” also includes multiple condensed ring systems (e.g. ring systems comprising 2, 3 or 4 rings) wherein a single heterocycle ring (as defined above) can be condensed with one or more heterocycles (e.g. decahydronapthyridinyl), carbocycles (e.g. decahydroquinolyl) or aryls. The rings of a multiple condensed ring system can be connected to each other via fused, Spiro and bridged bonds when allowed by valency requirements. It is to be understood that the point of attachment of a multiple condensed ring system (as defined above for a heterocycle) can be at any position of the multiple condensed ring system including a heterocycle, aryl and carbocycle portion of the ring. It is also to be understood that the point of attachment for a heterocycle or heterocycle multiple condensed ring system can be at any suitable atom of the heterocycle or heterocycle multiple condensed ring system including a carbon atom and a heteroatom (e.g. a nitrogen). Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl and 1,4-benzodioxanyl.

The term “carbocycle” or “carbocyclyl” refers to a single saturated (i.e., cycloalkyl) or a single partially unsaturated (e.g., cycloalkenyl, cycloalkadienyl, etc.) ring having 3 to 7 carbon atoms (i.e. (C₃-C₇)carbocycle). The term “carbocycle” or “carbocyclyl” also includes multiple condensed ring systems (e.g. ring systems comprising 2, 3 or 4 carbocyclic rings). Accordingly, carbocycle includes multicyclic carbocyles having 7 to 12 carbon atoms as a bicycle, and up to about 20 carbon atoms as a polycycle. Multicyclic carbocyles can be connected to each other via a single carbon atom to form a spiro connection (e.g. spiropentane, spiro[4,5]decane, spiro[4.5]decane, etc), via two adjacent carbon atoms to form a fused connection such as a bicyclo[4,5], [5,5], [5,6] or [6,6] system, or 9 or 10 ring atoms arranged as a bicyclo[5,6] or [6,6] system (e.g. decahydronaphthalene, norsabinane, norcarane) or via two non-adjacent carbon atoms to form a bridged connection (e.g. norbornane, bicyclo[2.2.2]octane, etc). The “carbocycle” or “carbocyclyl” can also be optionally substituted with one or more (e.g. 1, 2 or 3) oxo groups. Non-limiting examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl and 1-cyclohex-3-enyl.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of formula I, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

Specific values listed below for radicals, substituents, and ranges, are for illustration only; they do not exclude other defined values or other values within defined ranges for the radicals and substituents. The specific values listed below are specific values for compounds of formula I as well as all sub-formulas of formula I (e.g. formula Ia, Ib, Ic, Id).

Specifically, (C₁-C₆)alkyl can be methyl, ethyl, propyl, isopropyl, butyl, iso-butyl, sec-butyl, pentyl, 3-pentyl, or hexyl; (C₃-C₇)carbocycle can be cyclopropyl, cyclobutyl, cyclopentyl, cyclohexyl or cycloheptyl.

A specific group of compounds of formula I are compounds of formula Ia:

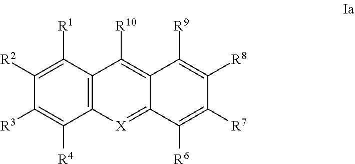

or a salt thereof.

Another specific group of compounds of formula I are compounds of formula Ib:

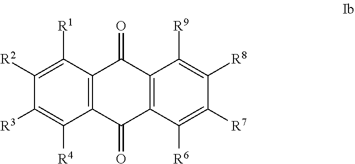

or a salt thereof.

Another specific group of compounds of formula I are compounds of formula Ic:

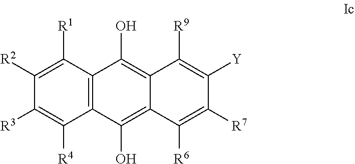

wherein Y is phenyl optionally substituted with one or more Z¹ groups, or a salt thereof.

Another specific group of compounds of formula I are compounds of formula Id:

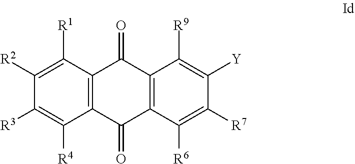

wherein Y is phenyl optionally substituted with one or more Z¹ groups, or a salt thereof.

A specific value for X is CR⁵.

A specific value for R⁸ is aryl, heteroaryl or heterocycle, wherein aryl, heteroaryl or heterocycle is optionally substituted with one or more Z¹ groups.

Another specific value for R⁸ is aryl, wherein is optionally substituted with one or more Z¹ groups.

In one embodiment the compounds of formula I do not include a compound of formula II:

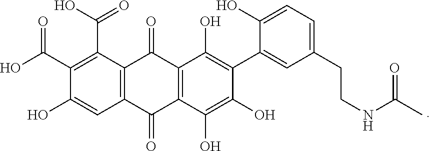 [SMILES: CC(=O)NCCc1ccc(O)c(-c2c(O)c(O)c3c(c2O)C(=O)c2c(cc(O)c(C(=O)O)c2C(=O)O)C3=O)c1]

In other embodiments, the methods of the invention use, and compositions of the invention comprise, compounds of formula III-XI:

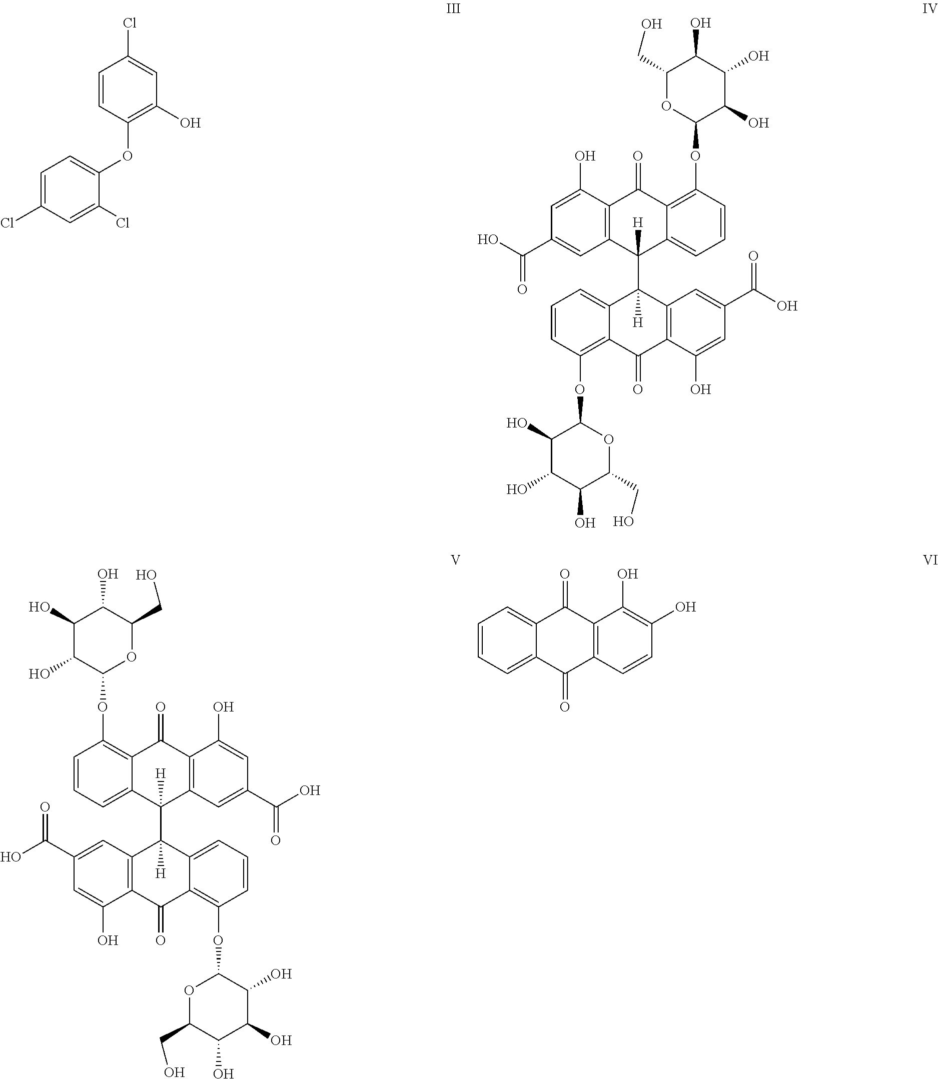

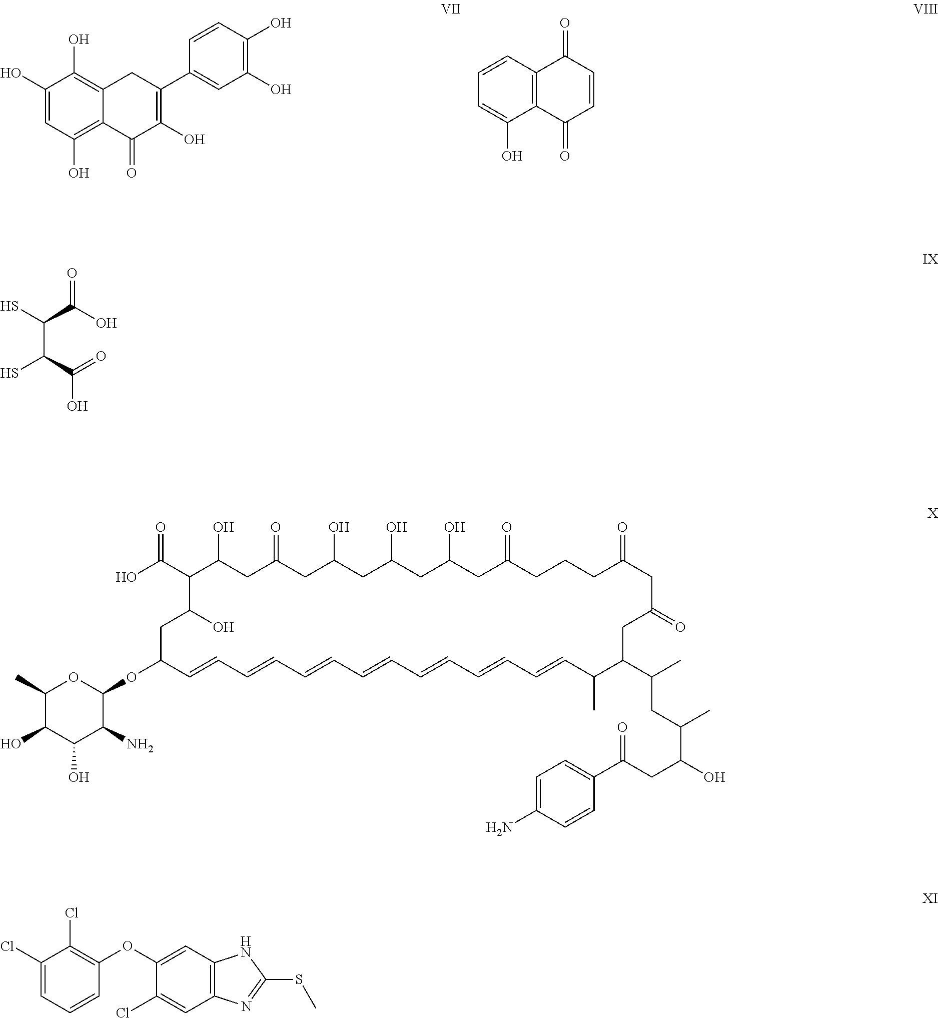 [SMILES: O=C1C(O)=C(c2ccc(O)c(O)c2)Cc2c(O)c(O)cc(O)c21 ; O=C1C=CC(=O)c2c(O)cccc21 ; O=C(O)[C@@H](S)[C@@H](S)C(=O)O ; CC1/C=C/C=C/C=C/C=C/C=C/C=C/C=C/C(O[C@@H]2O[C@H](C)[C@H](O)[C@@H](O)[C@@H]2N)CC(O)C(C(=O)O)C(O)CC(=O)CC(O)CC(O)CC(O)CC(=O)CCCC(=O)CC(=O)CC1C(C)CC(C)C(O)CC(=O)c1ccc(N)cc1 ; CSc1nc2cc(Cl)c(Oc3cccc(Cl)c3Cl)cc2[nH]1]

In cases where compounds are sufficiently basic or acidic, a salt of a compound of formula I-XI can be useful as an intermediate for isolating or purifying a compound of formula I-XI. Additionally, administration of a compound of formula I-XI as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula I-XI can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I-XI to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I-XI can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician. In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of formula I-XI formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful for the cancer. Examples of such agents include conventional cytotoxic chemotherapeutic agents or molecularly targeted drugs. Accordingly, in one embodiment the invention also provides a composition comprising a compound of formula I-XI, or a pharmaceutically acceptable salt thereof, at least one other therapeutic agent, and a pharmaceutically acceptable diluent or carrier. The invention also provides a kit comprising a compound of formula I-XI, or a pharmaceutically acceptable salt thereof, at least one other therapeutic agent, packaging material, and instructions for administering the compound of formula I-XI or the pharmaceutically acceptable salt thereof and the other therapeutic agent or agents to an animal to treat cancer.

In one embodiment of the invention the compounds formula I-XI inhibit one or more isozymes of DNMT. In another embodiment of the invention the compounds formula I-XI selectively inhibit one or more isozymes of DNMT in comparison to other isozymes of DNMT.

The ability of a compound of formula I-XI to act as an inhibitor of DNMT1 activity may be determined using pharmacological models which are well known to the art, or using Test A described below.

### Test A.

A Glal restriction endonuclease-coupled DNA methylation assay was used to assess Dnmt1 activity (Syeda et al, J Biol Chem 2011, 286, 15344-15351). Upon methylation of the DNA substrate by Dnmt1, the DNA substrate is cleaved by Glal, freeing a 5′ fluorophore from the 3′ quencher, and generating a fluorescence signal. Inhibition of Dnmt1 results in a loss of fluorescence generation.

Experimental results from Test A for a representative compound of formula I is described in Example 1. These results demonstrate that compounds of the invention are inhibitors of Dnmt1. Accordingly, compounds of the invention may be useful as therapeutic agents for the treatment of cancer including malignancies of the blood system (e.g. myelodysplatic syndromes) or solid tumors. Additionally, compounds of the invention may be useful as pharmacological tools for the further investigation of cancer (e.g. malignancies of the blood system including myelodysplatic syndromes or solid tumors) and cancer cell function.

The invention will now be illustrated by the following non-limiting Examples.

### Example 1

### Biological Evaluation of the Compounds of Formula II (Laccaic Acid A) to Formula XI

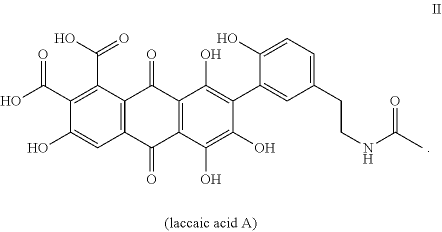 [SMILES: CC(=O)NCCc1ccc(O)c(-c2c(O)c(O)c3c(c2O)C(=O)c2c(cc(O)c(C(=O)O)c2C(=O)O)C3=O)c1]

Laccaic acid A was identified as a hit in a high throughput screen of the Spectrum collection (Microsource, Gaylordsville, Conn.) in a screen for inhibitors of Dnmt1 activity. A Glal restriction endonuclease-coupled DNA methylation assay to assess Dnmt1 activity (Syeda et al, J Biol Chem 2011, 286, 15344-15351). Upon methylation of the DNA substrate by Dnmt1, the DNA substrate is cleaved by Glal, freeing a 5′ fluorophore from the 3′ quencher, and generating a fluorescence signal. Inhibition of Dnmt1 results in a loss of fluorescence generation.

Laccaic acid A was further validated as a Dnmt1 inhibitor in three assays. First, laccaic acid was screened against the methyl-specific restriction endonuclease Glal (SibEnzyme, West Roxbury, Mass.) to ensure that the compound was not inhibiting Glal rather than Dnmt1. Under all conditions tested, Glal had full activity in the presence of laccaic acid A. Next, the concentration dependence of laccaic acid A inhibition on Dnmt1 activity was assessed. Increasing concentrations of laccaic acid A were used in the endonuclease-coupled DNA methylation assay to determine an IC₅₀ value for the compound. Laccaic acid A was determined to have an IC₅₀ value of 400 nM. Under identical conditions, the known Dnmt1 inhibitor SG14027 (Data, et al. Cancer Res 2009; 69, 4277-4285) has an IC₅₀ of 2 microM, indicating that laccaic acid A is 5-fold more potent at inhibition of Dnmt1. Lastly, differential scanning fluorimetry was used to determine if laccaic acid A was a direct binder of Dnmt1 in the absence of the DNA or S-adenosyl methionine substrate. The addition of laccaic acid A resulted in an increase in the observed melting temperature of Dnmt1 from 44° C. to 45.7° C., indicating that laccaic acid A was binding directly to Dnmt1 and stabilizing the protein against thermal denaturation. Table 1 shows Dnmt1 inhibition activity in the Glal restriction endonuclease-coupled DNA methylation assay for compounds II-XI.

TABLE 1

| Compound | IC₅₀ (μM) | 95% CI |
|---|---|---|
| III (Triclosan) | 3.8 | 2.4-5.9 |
| IV (Sennoside A) | 0.45 | 0.30-0.70 |
| X (Candicidin) | 0.4 | 0.27-0.60 |
| VI (Alizarin) | 11 | 7.9-15 |
| XI (Triclabendazole) | 6.1 | 3.4-11 |
| VII (Gossypetin) | 2.9 | 1.7-5.0 |
| V Sennoside B) | 0.45 | 0.33-0.60 |
| II (Laccaic Acid A) | 2.1 | 1.7-2.6 |
| VIII (Juglone) | 1.7 | 1.1-2.5 |
| IX (2,3-Dimercapto- | 0.33 | 0.22-0.51 |
| succinic Acid) | | |

### Example 2

The following illustrate representative pharmaceutical dosage forms, containing a compound of formula I-XI (“Compound Z”), for therapeutic or prophylactic use in humans.

| (i) Tablet 1 | mg/tablet |
|---|---|
| Compound Z | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound Z | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound Z | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml | |
|---|---|---|
| Compound Z (free acid form) | 1.0 | |
| Dibasic sodium phosphate | 12.0 | |
| Monobasic sodium phosphate | 0.7 | |
| Sodium chloride | 4.5 | |
| 1.0N Sodium hydroxide solution | q.s. | |
| (pH adjustment to 7.0-7.5) | | |
| Water for injection | q.s. ad 1 | mL |

| (v) Injection 2 (10 mg/ml) | mg/ml | |
|---|---|---|
| Compound Z (free acid form) | 10.0 | |
| Monobasic sodium phosphate | 0.3 | |
| Dibasic sodium phosphate | 1.1 | |
| Polyethylene glycol 400 | 200.0 | |
| 1.0N Sodium hydroxide solution | q.s. | |
| (pH adjustment to 7.0-7.5) | | |
| Water for injection | q.s. ad 1 | mL |

| (vi) Aerosol | mg/can |
|---|---|
| Compound Z | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula I: 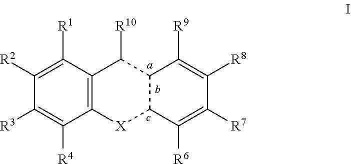
wherein:
X is N or CR⁵, the dashed bonds labeled a and c are double bonds and the dashed bond labeled b is a single bond; or X is CR⁵, the dashed bonds labeled a and c are single bonds, the dashed bond labeled b is a double bond and the substituents R⁵ and R¹⁰ are oxo (═O) groups;
R¹ is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more Z¹ groups;
R² is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more Z¹ groups;
R³ is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more Z¹ groups;
R⁴ is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more Z¹ groups;
R⁵ is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more Z¹ groups;
R⁶ is H, halo, (Cⱼ—C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more Z¹ groups;
R⁷ is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more Z¹ groups;
R⁸ is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more Z¹ groups;
R⁹ is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more Z¹ groups;
R¹⁰ is H, halo, (C₁-C₆)alkyl, —O(C₁-C₆)alkyl, (C₃-C₇)carbocycle, NRₐR_{b}, OH, CO₂H, aryl, heteroaryl or heterocycle, wherein aryl, heteroaryl or heterocycle is optionally substituted with one or more Z¹ groups;
each Z¹ is independently selected from halo, (C₁-C₆)alkyl, —O(C₁-C₆)alkyl, NRₐR_{b}, OH and CO₂H, wherein (C₁-C₆)alkyl is optional substituted with one or more groups selected from halo, —O(C₁-C₆)alkyl, NR_{c}R_{d}, NRₑC(═O)R_{f}, OH and CO₂H;
Rₐ and R_{b} are each independently H or (C₁-C₆)alkyl, or Rₐ and R_{b} together with the nitrogen to which they are attached form a heterocycle;
R_{c} and R_{d} are each independently H or (C₁-C₆)alkyl, or R_{c} and R_{d} together with the nitrogen to which they are attached form a heterocycle;
each Rₑ is independently H or (C₁-C₆)alkyl; and
each R_{f} is independently H or (C₁-C₆)alkyl;
or a salt thereof,
provided that said compound is other than laccaic acid A.

2. The compound of claim 1 which is a compound of formula Ia: 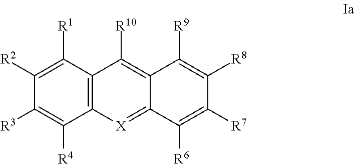
or a salt thereof.

3. The compound of claim 1 which is a compound of formula Ib: 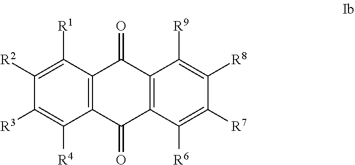
or a salt thereof.

4. The compound of claim 1 wherein R⁸ is aryl, heteroaryl or heterocycle, wherein aryl, heteroaryl or heterocycle is optionally substituted with one or more Z¹ groups.

5. The compound of claim 1 which is a compound of formula Id: 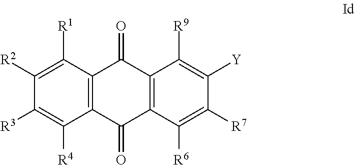
wherein Y is phenyl optionally substituted with one or more Z¹ groups, or a salt thereof.

6. The compound of claim 1 which is not a compound of formula II.

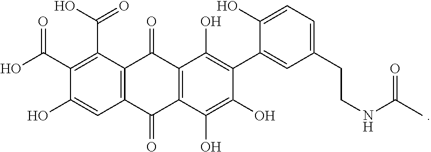 [SMILES: CC(=O)NCCc1ccc(O)c(-c2c(O)c(O)c3c(c2O)C(=O)c2c(cc(O)c(C(=O)O)c2C(=O)O)C3=O)c1]

7. A pharmaceutical composition comprising a compound of formula I as described in claim 1 or a compound of formula III-XI, 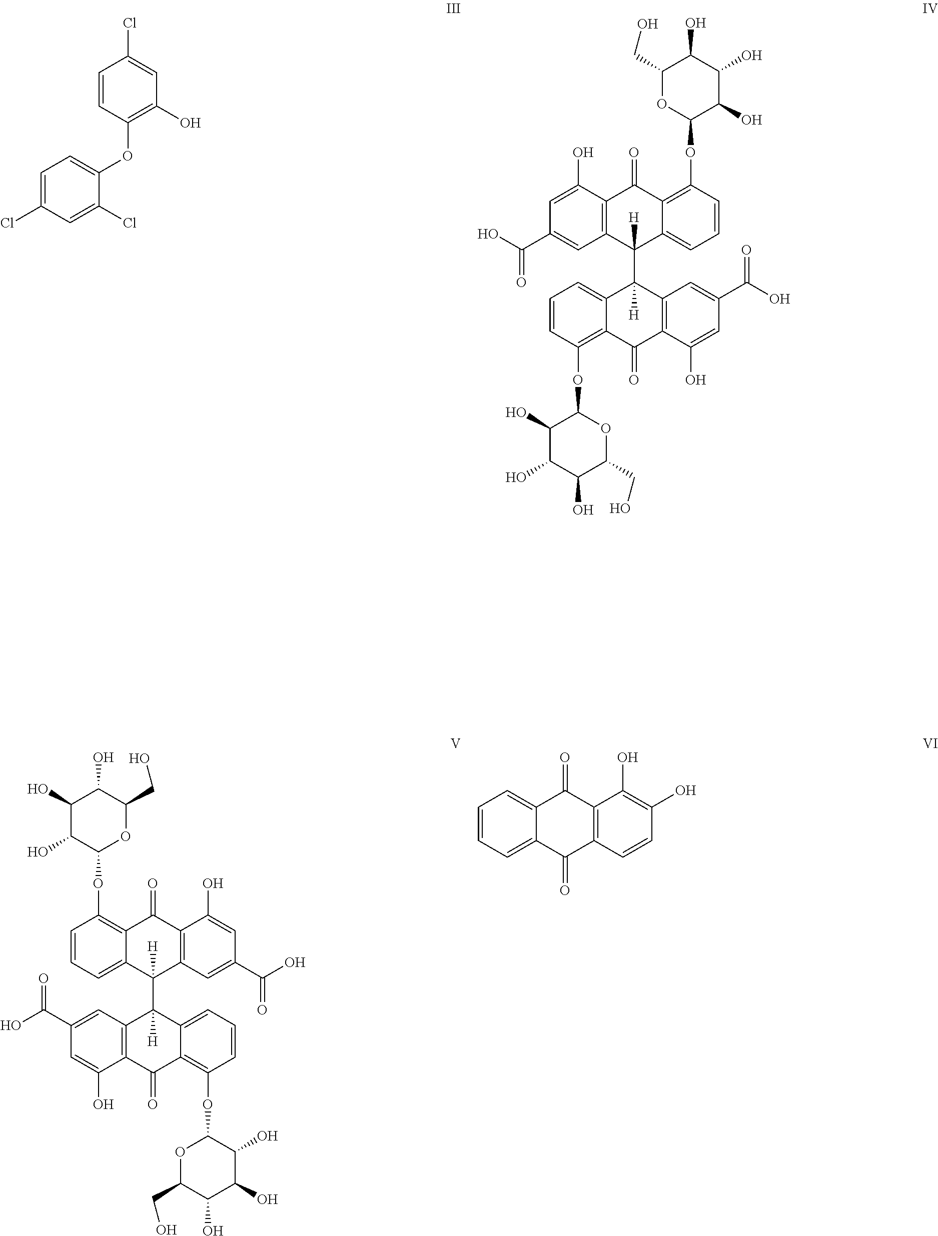 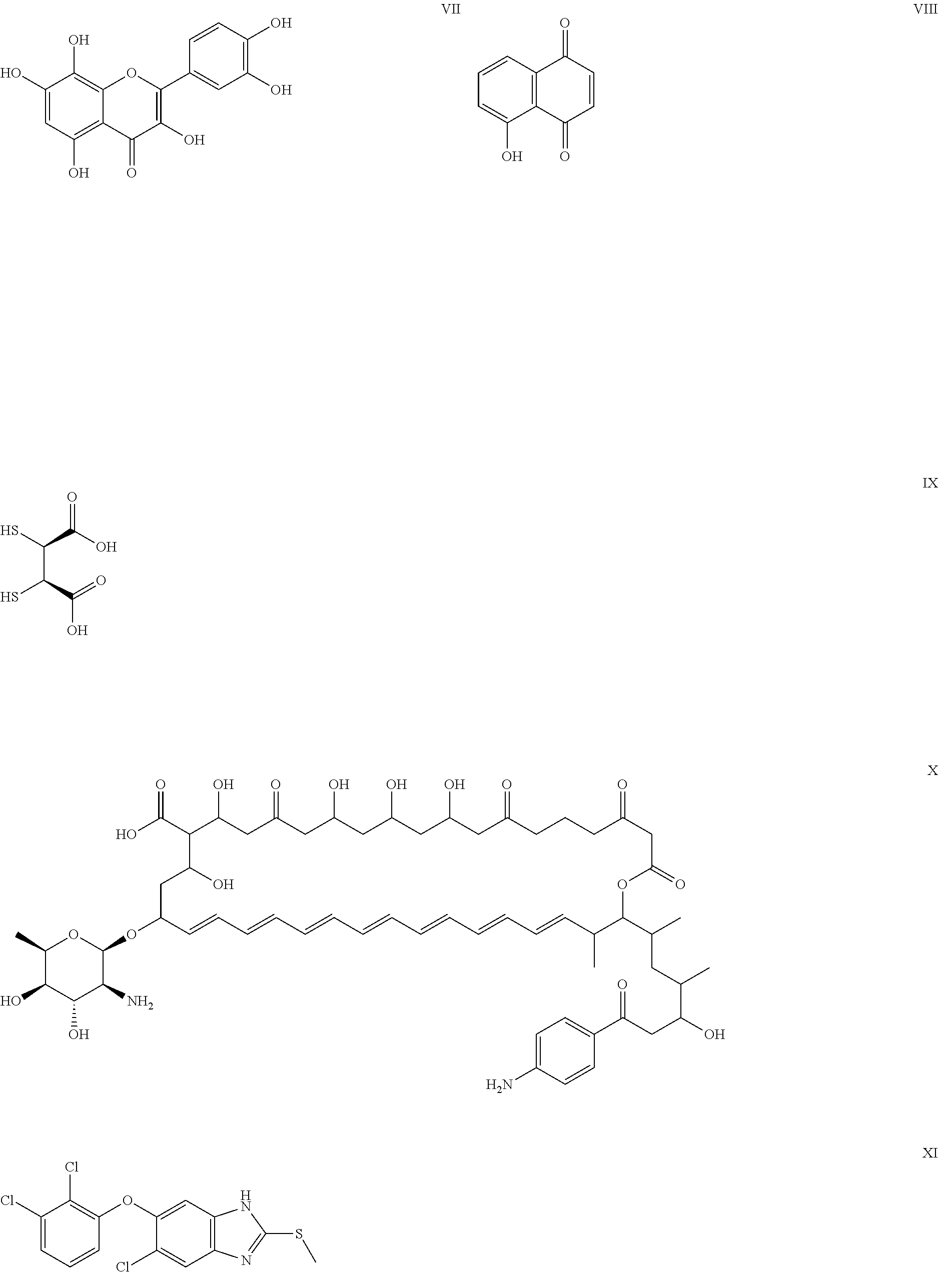 [SMILES: O=c1c(O)c(-c2ccc(O)c(O)c2)oc2c(O)c(O)cc(O)c12 ; O=C1C=CC(=O)c2c(O)cccc21 ; O=C(O)[C@@H](S)[C@@H](S)C(=O)O ; CC1/C=C/C=C/C=C/C=C/C=C/C=C/C=C/C(O[C@@H]2O[C@H](C)[C@H](O)[C@@H](O)[C@@H]2N)CC(O)C(C(=O)O)C(O)CC(=O)CC(O)CC(O)CC(O)CC(=O)CCCC(=O)CC(=O)OC1C(C)CC(C)C(O)CC(=O)c1ccc(N)cc1 ; CSc1nc2cc(Cl)c(Oc3cccc(Cl)c3Cl)cc2[nH]1]
or a pharmaceutically acceptable salt thereof, and a pharmaceutically acceptable carrier.

8. A method for suppressing cancer cell growth comprising contacting a cancer cell with a compound of formula I as described claim 1 or a compound of formula II-XI, or a salt thereof.

9. A method for inhibiting DNMT in a cell, comprising contacting the cell in vitro or in vivo with an effective amount of a compound of formula I as described claim 1 or a compound of formula II-XI or a salt thereof.

10. A method for treating cancer in a mammal comprising administering the mammal an effective amount of a compound of formula I as described in claim 1 or a compound of formula II-XI or a pharmaceutically acceptable salt thereof.

11. A pharmaceutical composition comprising a compound of formula II-XI: 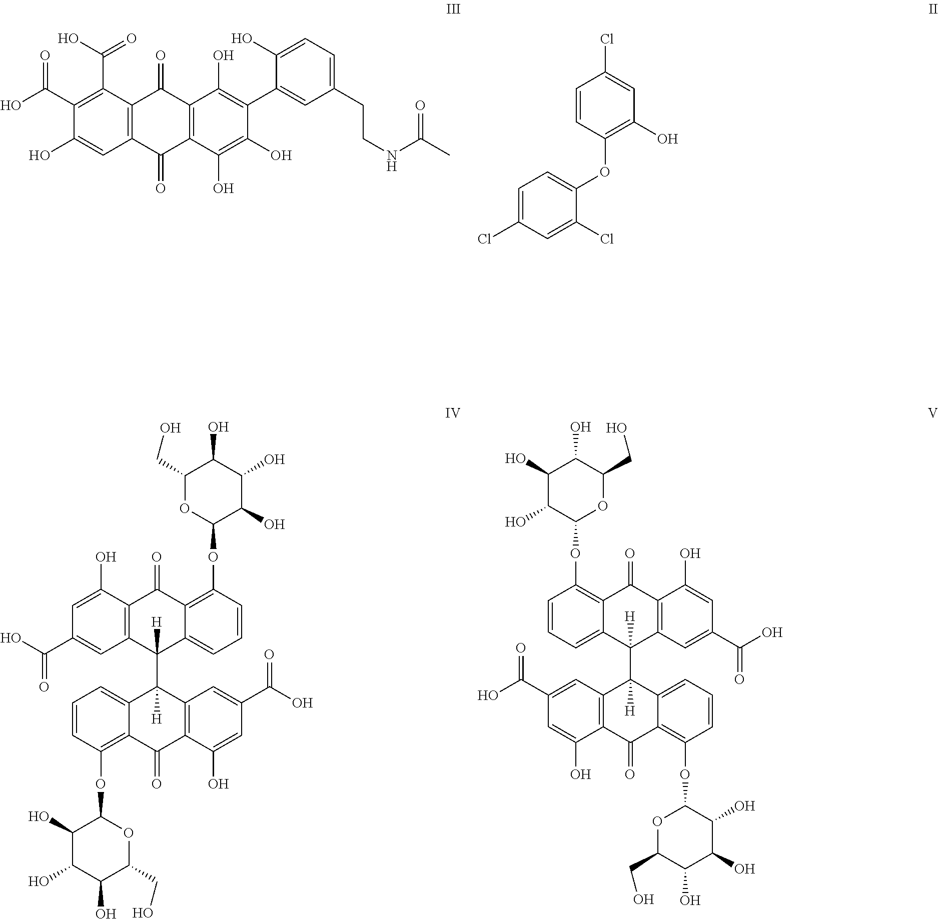 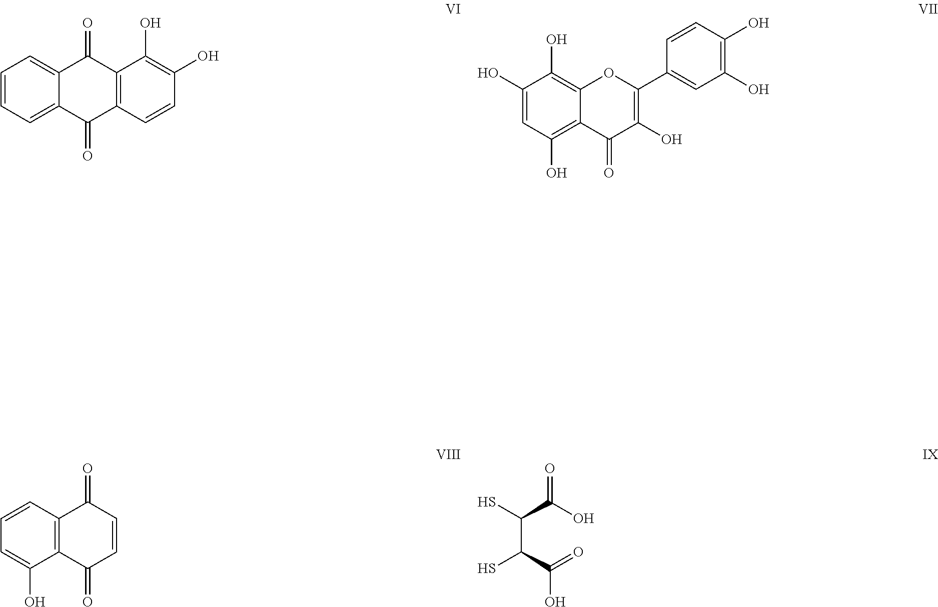 [SMILES: O=C1c2ccccc2C(=O)c2c1ccc(O)c2O ; O=c1c(O)c(-c2ccc(O)c(O)c2)oc2c(O)c(O)cc(O)c12 ; O=C1C=CC(=O)c2c(O)cccc21 ; O=C(O)[C@@H](S)[C@@H](S)C(=O)O] 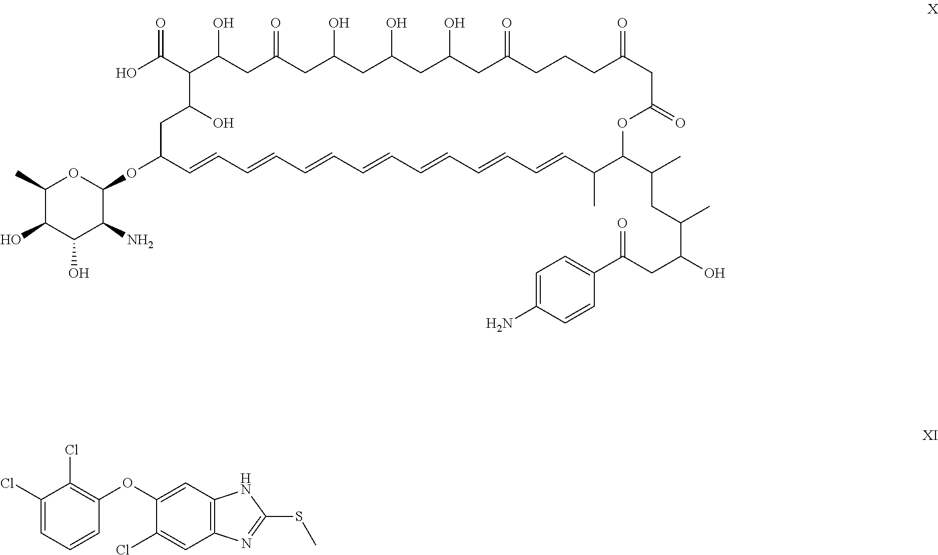 [SMILES: CC1/C=C/C=C/C=C/C=C/C=C/C=C/C=C/C(O[C@@H]2O[C@H](C)[C@H](O)[C@@H](O)[C@@H]2N)CC(O)C(C(=O)O)C(O)CC(=O)CC(O)CC(O)CC(O)CC(=O)CCCC(=O)CC(=O)OC1C(C)CC(C)C(O)CC(=O)c1ccc(N)cc1 ; CSc1nc2cc(Cl)c(Oc3cccc(Cl)c3Cl)cc2[nH]1]
or a pharmaceutically acceptable salt thereof, and a pharmaceutically acceptable carrier.

12. A method for suppressing cancer cell growth comprising contacting a cancer cell with a compound of formula II-XI as described in claim 11 or a salt thereof.

13. A method for inhibiting DNMT in a cell, comprising contacting the cell in vitro or in vivo with an effective amount of a compound of formula II-XI as described in claim 11 or a salt thereof.

14. A method for treating cancer in a mammal comprising administering the mammal an effective amount of a compound of formula II-XI as described in claim 11 or a pharmaceutically acceptable salt thereof.
